(19) United States
(12) Patent Application Publication
(10) Pub. No.: US 2023/0406908 A1
(43) Pub. Date: Dec. 21, 2023

(54) Methods of using passive immunity to decrease transmission of infectious diseases

(71) Applicant: CAMAS INCORPORATED, Le Center, MN (US)

(72) Inventors: Bradley M. Mitteness, Ghent, MN (US); Scott Simplot, Boise, ID (US); Michelle L. Hawkins, Bloomington, MN (US); Brian Robley, Mankato, MN (US); Philip Larson, Mankato, MN (US); Jason Hull, Wyoming, MN (US)

(21) Appl. No.: 18/032,491

(22) PCT Filed: Oct. 18, 2021
(86) PCT No.: PCT/US2021/055415

§ 371 Date: Apr. 18, 2023

Related U.S. Application Data
(60) Provisional application No. 63/093,598, filed on Oct. 19, 2020.

(51) Int. Cl. C07K 16/10

(52) U.S. Cl. CPC C07K 16/1003; A61K 2039/541; C07K 2317/76

(57) ABSTRACT

Egg laying birds on egg farms are inoculated with one or more disease antigens from microbial organisms causing infectious diseases. Egg material from the eggs contain one or more avian antibodies against the microbial organisms and these avian antibodies can specifically bind and/or neutralize the microbial organisms upon consumption by individuals in a population. Egg products comprising the one or more avian antibodies are provided to individuals in the population to increase the prevalence of avian antibodies against the microbial organism in the population. The egg products are provided in a variety of formulations for protecting individuals against the disease and/or for reducing the susceptibility for acquiring the disease. Egg products effective against infections of SARS-CoV-2 in individuals and for reducing the incidence of Covid-19 are provided. Methods are provided to decrease the infection rates of infectious diseases in a population of individuals.

### FIELD

The present description relates to infectious diseases and more particularly to methods of decreasing transmission of infectious diseases by use of avian antibodies for passive immunity.

### BACKGROUND

Widespread outbreaks of new pathogenic infections can have significant health and economic costs. The ability to prevent and/or reduce the diseases caused by the pathogenic organisms, especially using low-cost therapies that are easily administered, can be advantageous in developing countries as well as the whole world.

Severe acute respiratory syndrome coronavirus-2 (SARS-CoV-2), the virus strain that causes coronavirus disease 2019 (COVID-19), a respiratory illness emerged rapidly and led to a pandemic with huge world-wide health, economic and social consequences. SARS-CoV-2 is a positive-sense single-stranded RNA virus. Transmission of SARS-CoV-2 can occur by human-to-human transmission and thus humans in constrained spaces can lead to increase in transmission rates. Transmission occurs primarily via respiratory droplets from coughs and sneezes within a range of about 1.8 meters (6 ft). The virus may also be transmitted as aerosol particles. The virus is inactivated by soap, which destabilizes its lipid bilayer.

### SUMMARY

In one aspect, the present description relates to a method of reducing the prevalence of infectious diseases caused by one or more microbial organisms. The method includes administering an inoculum to female, egg laying birds on egg farms. The inoculum includes one or more disease antigens from the microbial organisms and collecting eggs laid by the inoculated female birds on the egg farms. The egg material from the eggs includes one or more avian antibodies that specifically bind and neutralize the microbial organisms. The method includes providing egg products that include the one or more avian antibodies to individuals susceptible to and/or having the infectious disease. The one or more avian antibodies in the egg products specifically bind and neutralize the microbial organisms in the individuals when consumed by the individuals.

The method may include that the one or more microbial organisms are bacteria, viruses, fungi, protozoa, worms or combinations thereof. The microbial organism may be a virus and the one or more avian antibodies may prevent a viral fusion molecule in the virus from attaching to the cellular attachment site of cells in the individual. The cellular attachment site in the cells may include the ACE2 receptor in the individual. The avian antibodies may be directed against an antigen from SARS-CoV-2, wherein the antigen may include s2 spike protein, s1 spike protein, spike rbd protein, nucleocapsid, envelope or combinations thereof. The avian antibodies may reduce the susceptibility of the individual to the disease upon consumption of the egg product.

The avian antibodies may reduce the symptoms in the individual from the disease upon consumption of the egg product. The egg product may be consumed by the individual at least once a day. The egg product may be consumed by the individual at least once a week. The egg product may be consumed by the individual more than once a day. The egg product may be whole eggs separated from the egg shells and at least part or all of the egg yolk includes functional antibody material when consumed by the individual. The egg product may be formulated for consumption by an individual and wherein the formulation comprises the one or more avian antibodies that bind and neutralize the microbial organisms when consumed by the individual. The egg product may be formulated for application to the pharyngeal area of the individual. The egg product may be administered to the nasopharyngeal area. The egg product may be administered to the oropharyngeal area.

The microbial organisms include one or more viruses. The one or more viruses may include Influenza viruses, Coronaviruses, Henipavirus, Ebola virus, Hantaan virus, Lassa fever virus, Marburg virus, Crimean-Congo haemorrhagic fever virus, Monkeypox virus, Rift Valley Fever virus, South American haemorrhagic fever viruses, Central European tick-borne encephalitis virus, Far Eastern tick-borne encephalitis virus, Japanese encephalitis virus, Russian spring and summer encephalitis virus, Kyasanur forest disease virus, Omsk hemorrhagic fever virus, West Nile virus, human respiratory syncytial virus (RSV), human metapneumovirus (hMPV), Picornaviridae, rhinoviruses, enteroviruses or combinations thereof. The one or more virus include viruses from the family Coronaviridae. The virus may include SARS-CoV-2. The consumption of the egg material may reduce susceptibility to Covid-19 or prevents Covid-19. The egg product may include egg powder. The egg product may be a nasal inhalant, a nasal spray, oral spray, mouthwash, oral tablet, a mint, chewing gum, a throat lozenge and combinations thereof.

In a further aspect, the present description relates to a method of decreasing the transmission rate of an infectious disease within a population of individuals. The method includes providing egg products comprising egg material from eggs to individuals in a population susceptible to or having the infectious disease. The eggs are from female birds inoculated with one or more disease antigens from microbial organisms causing the infectious disease. The egg material comprises one or more avian antibodies that specifically bind and neutralize the microbial organisms in the individuals when consumed by the individuals

The egg material may be formulated into a formulation comprising egg powder. The egg product may be a nasal inhalant, a nasal spray, mouth wash, oral spray, oral tablet, a mint, chewing gum, a throat lozenge and combinations thereof. The microbial organisms may be bacteria, viruses, fungi, protozoa, worms or combinations thereof. The microbial organism may be a virus. The one or more avian antibodies may prevent a viral fusion molecule in the virus from attaching to the cellular attachment site of cells in the individual. The cellular attachment site in the cells may include the ACE2 receptor in the individual. The avian antibodies may be directed against an antigen from SARS-CoV-2. The antigen may include s2 spike protein, s1 spike protein, spike rbd protein, nucleocapsid, envelope or combinations thereof.

The avian antibodies may reduce the susceptibility to the disease upon consumption of the egg product. The antibodies may reduce the symptoms from the disease upon consumption of the egg product. The egg product may be consumed by the individual at least once a day. The egg product may be consumed by the individual at least once a week. The egg product may be consumed by the individual more than once a day. The egg product may be egg material separated from the egg shells and at least part or all of the egg yolk in the egg material may include antibody material when consumed by the individual. The egg material may be formulated as an egg product for consumption by an individual. The formulation may include the one or more antibodies that bind and neutralize the microbial organisms when consumed by the individual. The egg product may be formulated for application to the pharyngeal area of the individual. The formulation may be administered to the nasopharyngeal area. The formulation may be administered to the oropharyngeal area.

The microbial organisms may include one or more viruses. The one or more viruses may include Influenza viruses, Coronaviruses, Henipavirus, Ebola virus, Hantaan virus, Lassa fever virus, Marburg virus, Crimean-Congo haemorrhagic fever virus, Monkeypox virus, Rift Valley Fever virus, South American haemorrhagic fever viruses, Central European tick-borne encephalitis virus, Far Eastern tick-borne encephalitis virus, Japanese encephalitis virus, Russian spring and summer encephalitis virus, Kyasanur forest disease virus, Omsk hemorrhagic fever virus, West Nile virus, human respiratory syncytial virus (RSV), human metapneumovirus (hMPV), Picornaviridae, rhinoviruses, enteroviruses or combinations thereof. The one or more virus may include viruses from the family Coronaviridae. The virus may include SARS-CoV-2. The consumption of the egg product may reduce susceptibility to Covid-19 or prevents Covid-19. The egg products are provided to all of the individuals in a population residing and/or working in a region with an outbreak of the infectious disease. The method may disrupt and/or decrease the transmission chain of the infectious disease from infected individuals in a population to uninfected individuals in a population. The individuals consuming the egg products may shed neutralized microbial organisms.

In yet a further aspect, the present description relates to a composition including one or more avian antibodies. The one or more avian antibodies specifically bind and neutralize disease-causing microbial organisms in individuals of a population when consumed by the individuals in a population. The one or more avian antibodies are generated by inoculation of egg-laying birds with one or more of the microbial organisms. The one or more avian antibodies may be formulated into a mint. The composition may be effective for shedding neutralized disease-causing organism by an infected individual when the infected individual consumes the one or more avian antibodies.

### BRIEF DESCRIPTION OF THE FIGURES

FIG. 1 is a plot depicting the absolute neutralization of virus observed in this assay at 1:10 dilution of the antibodies. Control refers to virus count in the absence of the antibody.

FIG. 2 is a plot depicting the same data as in FIG. 1 but as a relative percentage of neutralization observed in the context of the four antibodies as compared to the virus control.

### DEFINITIONS

Various terms are defined herein. The definitions provided below are inclusive and not limiting, and the terms as used herein have a scope including at least the definitions provided below.

The terms “preferred” and “preferably”, “example” and “exemplary” refer to embodiments that may afford certain benefits, under certain circumstances. However, other embodiments may also be preferred or exemplary, under the same or other circumstances. Furthermore, the recitation of one or more preferred or exemplary embodiments does not imply that other embodiments are not useful, and is not intended to exclude other embodiments from the inventive scope of the present disclosure.

The singular forms of the terms “a”, “an”, and “the” as used herein include plural references unless the context clearly dictates otherwise. For example, the term “a tip” includes a plurality of tips.

Reference to “a” chemical compound refers to one or more molecules of the chemical compound, rather than being limited to a single molecule of the chemical compound. Furthermore, the one or more molecules may or may not be identical, so long as they fall under the category of the chemical compound.

The terms “at least one” and “one or more of” an element are used interchangeably, and have the same meaning that includes a single element and a plurality of the elements, and may also be represented by the suffix “(s)” at the end of the element.

The terms “about” and “substantially” are used herein with respect to measurable values and ranges due to expected variations known to those skilled in the art (e.g., limitations and variability in measurements).

The term “and/or” means one or all of the listed elements or a combination of any two or more of the listed elements.

The terms “comprises,” “comprising,” and variations thereof are to be construed as open ended—i.e., additional elements or steps are optional and may or may not be present.

The term “microbial adherence inhibitor” as referred to herein includes molecules that bind and inhibit microbes, e.g. bacteria, viruses and the like.

The term “antibody” or “avian antibody” as referred to herein relates to immunoglobulin molecules and is one embodiment of a microbial adherence inhibitor. The embodiments are described herein with reference to antibodies but it will be understood that microbial adherence antibodies other than antibodies may also be used and all are within the scope of this description. Antibodies and avian antibodies will be used interchangeably.

The term “disease-causing organisms” as referred to herein relates to pathogenic organisms that include viruses, bacteria, fungi, protozoa, worms and combinations thereof.

The term “immunogens” as referred to herein includes disease-causing organisms and/or material derived from disease-causing organisms that are used as antigens for inoculating hens and generate an immune response in the hens against the disease-causing organisms.

The term “disease antigens” as referred to herein includes antigenic material from the disease-causing organism. The disease antigens can be whole organisms or material derived from the disease-causing organism.

The term “mucosal membrane” as referred to herein includes a membrane lining all body passages that communicate with the air, such as the respiratory and alimentary tracts, and having cells and associated glands that secrete mucus. Mucosal membranes can be found, for example, in mouth, nose, eyes, eyelids, trachea, lungs, stomach, intestines, and the like.

The term “pharyngeal area” as referred to herein includes the oropharynx, nasopharynx, and laryngopharynx of an individual.

The term “virus fusion molecule” or “viral fusion molecule” as referred to herein relates to the molecule or molecules on a virus that are involved in binding and/or attaching to the cells of the individual host.

The term “cellular attachment site” as referred to herein relates to a molecule, e.g. a receptor, on the host cells that is the site for viral attachment.

The term “neutralize” as referred to herein relates to binding by avian antibodies to a disease-causing organism, e.g. virus, and prevents the attachment of the disease-causing organism to the cellular attachment site in the cells of an individual.

The term “secondary infections” as referred to herein relate to infections caused by a disease-causing organism after a first disease-causing organism, e.g. a virus, has infected an individual. The individual can become susceptible to the secondary infection due to a weakened immune system as a result of the primary or first infection.

The term “functional antibodies” as referred to herein relate to antibodies that retain their function of binding to their target site and/or binding the target microbial organism.

The term “consumption” as referred to herein relates to internalization of egg products by the individual. Consumption can include ingesting the egg product such as a mint or a tablet, inhalation of powder through nasal passages, administration of nasal spray, oral spray, eye drops, infusion of the egg product and any other means of using the egg material to bind and neutralize the disease-causing organism in an individual.

The term “egg material” as referred to herein relates to egg whites and/or egg yolks from a bird inoculated with the one or more disease antigens. The egg whites and/or egg yolks are separated from the eggshells.

The term “egg product” as referred to herein relates to any product that includes egg material, e.g. egg yolks and/or egg whites, from eggs of birds inoculated with the one or more disease antigens. The egg product includes functional antibodies against the target disease causing organism(s). Egg product can also include functional antibodies derived from egg material such as purified, partially purified and/or unpurified antibodies derived from the egg material of birds inoculated with the one or more disease antigens. Egg products can also include spray-dried powder of the egg material and/or spray dried powder of one or more avian antibodies derived from the eggs described herein. Egg products also include formulations containing the egg material and/or avian antibodies derived from the egg material described herein. The formulations can be, for example, tablets, mints, lozenges, chewing gum, or other edible products. The formulations can also include nasal spray, oral spray, eye drops, mouth wash, antibody powder and the like that can be consumed and/or internalized by an individual for protection against the disease-causing organisms. Egg product and egg material may be used interchangeably in the description herein.

The term “infectivity rate” or “infection rate” as referred to herein relates to the number of individuals in a population that are infected compared to the whole population of individuals.

The term “transmission chain” as referred to herein relates to transmission of a disease-causing organism by an infected individual to an uninfected individual whereby the uninfected individual becomes infected due to exposure to the disease-causing organisms shed by the infected individual.

The term “passive immunity” as referred to herein relates to short-term immunity against a disease in a person or animal that results from the introduction of specific antibodies against the disease from another person or animal.

### DETAILED DESCRIPTION

The present description includes methods of managing infectious diseases caused by pathogenic microorganisms. The method includes decreasing the transmission rate of infectious diseases in a population by providing passive immunity to individuals of the population. The method includes managing infectious diseases by providing avian antibodies in egg products that are consumed by individuals in a population. The individuals in a population can consume egg product derived from eggs laid by a female bird inoculated with one or more disease antigens from the disease causing organisms. The method can include inoculating egg-laying birds on egg farms with one or more antigens from the disease-causing organisms. This can result in an egg supply having egg yolks that are enhanced with avian antibodies specific for binding and/or neutralizing the disease-causing organisms. The egg material from these eggs includes one or more avian antibodies against the disease-causing organisms. Egg products derived from the egg materials can include functional avian antibodies that are capable of specifically binding and/or neutralizing the disease-causing organisms when consumed by individuals in a population.

In one embodiment, an individual can consume egg yolks that are completely or partially uncooked, e.g. runny yolks. In one embodiment, the individuals can consume egg product that can include egg powder that includes avian antibodies from the egg material and wherein the egg powder is formulated for consumption and the antibodies in the egg powder include functional antibodies against the disease causing organisms. In one embodiment, consumption of the egg product can prevent infection in an individual susceptible to the infectious disease. In one embodiment, consumption of the egg product can reduce the symptoms of the infectious disease in an individual with the infectious disease. In one embodiment, consumption of the egg product can reduce and/or disrupt the transmission chain of the infectious disease from an infected individual to uninfected individuals.

In one embodiment, individuals in a population can consume egg yolks that are completely or partially uncooked, e.g. runny yolks. In one embodiment, individuals in a population can consume egg product that can include egg powder with avian antibodies from the egg material and wherein the egg powder is formulated for consumption and the antibodies in the egg powder include functional antibodies against the disease causing organisms. In one embodiment, consumption of the egg product can decrease the infection rate in the population of individuals.

In many viral diseases, the spread of the virus is greatest during the asymptomatic phase or the early symptomatic phase that is around and immediately following the onset of symptoms. In an individual. Often individuals can be exposed to respiratory viruses and risk being infected by being in the presence of infected individuals that are asymptomatic or have mild and/or non-specific symptoms. In one embodiment, the consumption of the egg product can disrupt and/or decrease the transmission of infectious diseases by the infectious agent from infected individuals to uninfected individuals in a population.

Exposure to respiratory viruses such as SARS-CoV-2 can lead to respiratory infections as a result of the entry of the virus through the mucous membranes in the pharyngeal areas when an individual is exposed to the virus. Without any mucosal protectant, the viruses can make the journey from the pharyngeal area to the lower respiratory tract to the interior of the lungs. This can lead to the respiratory diseases such as Covid-19. Transmission between humans can be through expelled viral particles in respiratory droplets, saliva, airborne droplets and the like. Once the microorganisms are established in the pharyngeal area, respiration can result in downward carriage of the viral pathogens into the lower respiratory tract. This allows the organisms to attach to the bronchi and alveolar cells and to multiply and increase viral load.

In some embodiments, the present description can include methods for protecting individuals in a population from emerging infectious diseases. In emerging infectious diseases, the infectious disease has newly appeared in a population or has existed but is rapidly increasing in incidence or geographic range. The rapid emergence can limit the ability of the scientific community to develop therapeutic drugs and/or vaccines in time to reduce the negative outcomes in a population. Moreover, prior art methods of generating vaccines against infectious diseases such as influenza can result in vaccines that only partially protect the individual receiving the vaccine. Generally, influenza vaccines are based on predictions of specific strain(s) that may be the most prevalent in the next infectious cycle. Success of the vaccines can be highly variable and often can be dependent on the successful prediction of the prevalent strains a year later resulting in vaccines that may not accurately correlate with the most prevalent strain(s) of the disease causing organisms. Fatalities and negative outcomes in a population can occur and at great economic and emotional costs.

The time from the onset of a pandemic to the creation of an effective vaccine is a difficult and crucial period. During this period, daily life can be altered to slow transmission by the means of social isolation, wearing masks, frequent testing, and isolation. This disruption can have significant economic and social costs. Isolation of human beings is an unnatural and difficult expectation to combat a public health crisis.

In the SARS-CoV-2 pandemic of 2020, the first response by governments world-wide was to ask the populations within each of the countries to follow governmental social distancing guidelines by the individuals in a population. This approach provided mixed results due to compliance issues, especially as the duration of the outbreak became longer resulting in fatigue and loosening of the adoption of the prescribed methods such as social distancing and mask wearing. This resulted in subsequent waves of outbreaks of the infectious disease.

Methods that fit instinctively within everyday life of a society can fill the void opened by fatigue of social distancing, etc. Such methods can be successful if they fit within the existing comfort zone of the population. The more rapid the adoption, the broader the adoption and the more disruption of the transmission chain of infection between individuals.

A method for rapidly generating and disseminating safe avian antibodies against disease causing organisms and providing passive immunity to individuals in a population in a cost-effective manner can be societally advantageous. In one embodiment, methods described herein can generate safe antibodies for use in providing passive immunity in about 3 to about 4 weeks after inoculation of the egg-laying birds. The rapid generation of the antibodies for passive immunity can enable rapid response to emerging infectious agents. The method also can enable rapid adaptation to mutations that may arise in the pathogenic organisms or to strains that are most prevalent in the community.

In addition, the methods described herein advantageously use egg materials for combating outbreaks of infectious diseases. Eggs are a common food and as such individuals in a population are comfortable ingesting eggs, egg materials and egg product formulations can lead to a higher degree of compliance than, for example, a new vaccine or continuous mask wearing in public spaces. In the public health arena, the degree of compliance by a population of individuals is significant for the success of the method in combating an infectious disease outbreak. Without being bound by any theory, it is believed that greater the compliance for a method by a population of individuals then the greater the success of the method. Furthermore, the compliance of a population of individuals is greater if they are comfortable with the therapy, e.g. a mint versus a vaccine. Compliance by a population of individuals can also be greater if the hurdles are lowered or if the ease of being compliant is reduced for the individuals.

In one exemplary embodiment, the egg product is a mint and populations of individuals are comfortable with taking a mint that includes egg materials for combating infectious disease outbreaks. In one embodiment, a method of providing a mint to, for example, a kindergartner has a greater likelihood of compliance than wearing a mask the entire school day. In other words, in terms of a carrot and stick approach to combat public health crisis, a population of individuals may view taking a mint as a “carrot” and having to take a vaccine or wear a face mask as a “stick”. This use of the “carrot” can increase the compliance of a population of individuals.

In some embodiments, the emerging infectious disease can be epidemic. In some embodiments, the emerging infectious disease can be pandemic.

In some embodiments, the individuals in a population can be animals, mammals, farm animals, humans and the like. In one embodiment, the individuals are humans and the population is the human population.

In the methods described herein, egg-laying birds on egg farms can be inoculated with the one or more disease antigens. The individuals in the population can be protected from the disease-causing organisms by consuming egg products derived from eggs laid by the inoculated birds. The egg products can include avian antibodies that specifically bind and neutralize the circulating disease-causing organisms in the individual upon consumption of the antibodies. The egg products that include avian antibodies from the egg yolks can be widely distributed to individuals and stored until use. In some embodiments, they can be self-administered. The egg products described herein can be all natural preparations that contain specific avian antibodies to the targeted immunogens. Consumption of the egg product(s) with functional avian antibodies can specifically bind and neutralize the targeted disease causing organisms.

In some embodiments, the egg products can include microbial adherence inhibitors, e.g. antibodies. The embodiments described herein will be discussed in the context of avian antibodies, but it will be understood that other microbial adherence inhibitors may be used and are within the scope of this description.

In some embodiments, the avian antibodies are derived from birds inoculated with the target immunogens, e.g. viral antigens, bacterial antigens and the like. Birds, in particular, have the ability to “load up” their eggs as they are formed, with a very large supply of antibodies. Once immunized, the hen deposits IgY type immunoglobulins in the yolk while depositing IgM and IgA immunoglobulins in the albumin. Thus, the egg yolks from the immunized hens are enriched or have enhanced supply of IgY antibodies directed to targeted disease-causing organism. The albumin helps add stability to the whole egg preparations and helps protect the avian antibodies. The avian IgY immunoglobulins in the yolk can tightly bind to, coat and cover the mucosal membranes in an individual to bind and inhibit the entry of the target virus particles. The IgM and IgA immunoglobulins in the albumin increase binding of the antibody containing material in the mucous membrane in the pharyngeal area. This can provide longer sustaining effect of the antibody containing material. The larger antibody containing molecules are more effective in preventing adherence of the targeted immunogen, e.g. virus, in the pharyngeal area of the human. Albumin is a protein that protects the activity of the IgY immunoglobulins thereby increasing their active life. Furthermore, a large fraction of the antibodies deposited in the eggs by the hen are against the most recent antigenic challenges to the hen. This all results in the eggs of birds being a most ideal source for large quantities of economically produced highly specific and stable antibodies. While the present description is illustrated by the use of chickens to produce avian antibodies, other fowl including turkeys, ducks, geese, ostrich, Emu, pheasant, pigeon, quail, etc. or combination thereof, may be used.

In addition to this direct attack by administered antibodies, components of the complement system included in most biological fluids, such as blood, lymph, saliva, tears and to some extent intestinal secretions, recognize an antibody attachment as triggers for their many types of defensive activities. Specific antibody attachment and coating combined with the very likely mobilization of many other cellular defense systems, therefore, quickly culminating in the chemical inactivation and ultimately the destruction of the targeted microorganism.

In some embodiments, the method of managing an infectious disease can include administering an inoculum that includes one or more disease antigens to female, egg laying birds on egg farms. In some embodiments, all of the female egg-laying birds at the egg farms are inoculated. In some embodiments, at least about 95%, or at least about 90%, or at least about 80%, or at least about 70% or at least about 60%, or at least about 50%, or at least about 25%, or at least about 10%, or at least about 5% of the birds are inoculated with the one or more disease antigens. Inoculating less than about 5% of the birds is also within the scope of the description.

In some embodiments, the inoculum can include one disease antigen and all the birds on an egg farm receiving an inoculum are inoculated with the same disease antigen. In some embodiments, the inoculum can include more than one disease antigen and all the birds on an egg farm receiving an inoculum are inoculated with the same disease antigens. In some embodiments, the inoculum can include one or more disease antigens and an egg farm can include groups of birds, wherein each group receives an inoculum of a different one or more disease antigens. The egg material from the birds receiving the different inoculums of one or more disease antigens can be combined and processed together or the egg material may be processed separately.

In some embodiments, the method can include collecting the eggs from the egg-laying hens inoculated with one or more disease antigens. The method can include providing whole eggs from the inoculated birds to the population. The method can also include providing egg products that contain egg material from the eggs collected from hens inoculated with the one or more disease antigens.

In one embodiment, the population of individuals may be provided egg products that are whole eggs in a supermarket or other establishment. Individuals buying the eggs can take the eggs home and prepare the eggs in a desired method for consumption. The preparation of the eggs for consumption can be done in a manner that maintains the function of the antibodies in the yolk. The eggs may be cooked for consumption with the yolk remaining runny. The eggs may be added to other food ingredients and consumed. Consumption of eggs is done in a manner that can retain the capability of the avian antibody to specifically bind and/or neutralize the targeted microbial organism.

In some embodiments, the method can also include providing egg products with the egg material derived from the eggs described herein to a population of individuals susceptible to the infectious disease and/or having the infectious disease. The egg products can include egg material and/or one or more avian antibodies that specifically bind and neutralize the targeted microbial organisms in an individual when consumed by the individual.

In some embodiments, the egg products may be formulated as described herein. In one embodiment, the egg materials, e.g. the egg yolks, egg whites, purified avian antibodies, partially purified avian antibodies, may be powder such as a spray dried powder, and consumed as a powder or further formulated into other egg products such as nasal spray, oral spray, tablets, mints, lozenges, mouth wash, chewing gum and the like.

In some embodiments, the method can include increasing the prevalence of the avian antibodies that specifically bind and neutralize the one or more disease-causing organisms in a population of individuals. The increase in the prevalence of the avian antibodies in the population of individuals against the targeted disease-causing organism can decrease the infection rate of transmission of the infectious disease amongst the individuals in the population and lead to a decrease in the prevalence of the infectious disease in the population.

In some embodiments, the method can include administration of the avian antibodies that specifically bind and neutralize the disease causing organisms to individuals of a population to decrease the transmission chain from infected individuals, who may be shedding the disease-causing organisms, e.g. the virus particles, to infect the uninfected or naive individuals in the population. The infected individuals may be symptomatic individuals and/or asymptomatic individuals. The method can decrease the disease causing organism load in a population of individuals and decrease the exposure of the amount of disease causing pathogen of a population of individuals.

In some embodiments, the method can include providing avian antibodies to all or most of the individuals in a population in a community with an outbreak of the infectious disease. In one embodiment, the whole population in an outbreak or a hotspot can be treated. Treating the whole population can reduce the pathogen load across the whole population and can lead to a reduction in exposure loads of the pathogenic organism by the population of individuals and a decrease in prevalence of the infectious disease. Advantageously, the methods described herein are simple, safe, inexpensive, massively scalable tools to decrease or eliminate the prevalence of the infectious disease. Advantageously, since the antibodies are shelf stable and need no special packaging or handling to be quickly delivered via a postal system. In addition, there are no dosage constraints since egg materials are food.

By all or most of the individuals in a population, it is meant that at least about 95% of the individuals of a population consume the egg products, or at least about 90% of the individuals of a population consume the egg products, or at least about 80% of the individuals of a population consume the egg products, or at least about 70% of the individuals of a population consume the egg products, or at least about 60% of the individuals of a population consume the egg products, or at least about 50% of the individuals of a population consume the egg products, or at least about 25% of the individuals of a population consume the egg products. Percent of individuals in a population outside of this range consuming the egg product are also within the scope of this description.

In some embodiments, greater than about 90% of the individuals in a population, or greater than about 95% of the individuals in a population, or greater than about 98% of the individuals in a population, greater than about 99% of the individuals in a population are provided the egg product formulations with the specific avian antibodies described herein. Without being bound by any theory, it is believed that greater the participation rate of the individuals in a population, the greater the benefit or success of the methods described herein.

In one embodiment, the avian antibodies are formulated as mints. The mints can be provided to the individuals in a population. In one embodiment, the avian antibodies are provided, for example, as mints and delivered to households by a delivery system, e.g. postal service, to a hotspot or site of the infectious disease outbreak. The delivery can be coordinated by a governmental agency or other community based organizations upon the knowledge of an imminent outbreak or during an outbreak.

In one embodiment, the avian antibodies can be delivered to a population of individuals via their address and using the post office. The amount of avian antibody delivered within a geographic area of outbreak can be adjusted for population density. For example, Manhattan can receive more avian antibody egg product than, for example, Sioux Falls, South Dakota within the same number of square blocks. A high density of individuals in the population can increase the number of individual opportunities of transmission events/day and thus providing increased amounts of avian antibodies to high density regions can decrease the number of transmission events/day.

In one embodiment, the avian antibodies are provided during the early stages of an outbreak. In one embodiment, the amount of avian antibodies provided to a household can be dependent on the size of the household and the amount is sufficient for all of the individuals of the household. In one embodiment, all of the individuals of the household, infected and uninfected, can consume the avian antibodies.

Advantageously, in the methods described herein, all of the individuals of the population in a known hot spot do not have to be continuously tested for the presence of the infectious organism. All or most of the individuals in the population can be treated without needing to identify and isolate the infected individuals. When all individuals of the geographic group are treated simultaneously, the symptomatic shedders, asymptomatic shedders and uninfected individuals ingest the avian antibodies that bind the disease-causing agent. The negative consequences of asymptomatic shedders in the population are decreased and the asymptomatic shedders may be present within the population without isolation. In one embodiment, the disease-causing organisms shed by the infected individuals can be neutralized disease-causing organisms due to the avian antibody bound to the disease-causing organism shed by an infected individual. Without being bound by any theory, it is thought that ingestion of the avian antibodies by infected individuals can lead to specific binding of the disease-causing organism by the avian antibody and can result in shedding of neutralized disease-causing organisms, e.g. cold viral particles, instead of infectious disease-causing organisms, e.g. hot viral particles. The neutralized disease-causing organisms are disease-causing organisms that are no longer infectious to uninfected or naïve individuals of the population, resulting in breaking the transmission chain between infected individuals and uninfected individuals.

The methods described herein can be advantageously used for providing avian antibodies for populations since generation of the avian antibodies is economical. In some embodiments, the egg material in each egg can include between about 1 mg and about 10 mg of specific avian antibodies against the disease-causing organism. In one embodiment, avian antibodies are formulated into mints and, for example, about 25 mints can be made from the egg material of one egg. In one embodiment, there are approximately between about 1×10¹⁴ and about 1×10¹⁶ antibody molecules per mint.

The number of doses administered per day of the egg products can vary and all are within the scope of this description. In one embodiment, at least about one dose per day of the egg products is administered. In some embodiments, at least about two doses per day, or at least about 3 doses per day, or at least about 4 doses per day, or at least about 5 doses per day, or at least about 6 doses per day or more may be administered. In some embodiments, at least about one dose a week, or at least about two doses per week, or at least about 3 doses per week, or at least about 4 doses per week, or at least about 5 doses per week, or at least about 6 doses per week or more may be administered.

Doses can be administered for any length of time. In some embodiments, the doses can be administered as long as the threat of a viral infection is present. Doses may be administered for about 1-7 days, or about 7 to 14 days, or about 14 to 21 days, or about 21-31 days, or about more than one month, or about more than two months, or about more than three months, or about more than 6 months, or about more than a year.

The amount of egg material in each dose can vary and all are within the scope of this description. In some embodiments, the amount of egg powder in a formulation can depend on the type of formulation, the excipients used and the like. In some embodiments, the amount of spray dried egg powder can include between about 1 mg and about 100 grams. Amounts outside of this range are also within the scope of this description. In some tablet or throat lozenge embodiments, the amount of egg material may be between about 0.1 grams and about 5 grams. In some gargle embodiments, the amount of egg material may be between about 1 gram and about 25 grams. In some nasal embodiments, the amount of egg material may be between about 1 mg and about 1 gram.

In one embodiment, the dose may be a whole soft-boiled egg. A soft-boiled egg can be enriched with the avian antibodies against the targeted disease-causing organism. The soft-boiled egg can include avian antibodies that have not been inactivated in the cooking process and that can coat the mucous membranes of the individual and provide a protective effect. An individual may consume one or more soft-boiled eggs per day, two or more soft-boiled eggs per day. The soft boiled egg(s) may be consumed one or more times a week, or two or more times a week, or three or more times a week, or four or more times a week, or five or more times a week, or six or more times a week.

In one embodiment, the methods described herein can include a public health initiative to inoculate birds on all farms to be inoculated with one or more disease antigens from the targeted disease-causing organism to generate egg products in the country that are protective against the emerging targeted disease-causing organism. The methods can also include a public health initiative to encourage individuals in a population to consume egg products as described herein. Individuals may consume the egg products for any length of time. In one embodiment, individuals may consume the egg products until the threat from the emerging infectious disease is over.

In one embodiment, the egg product can include purified avian antibodies against the targeted microbial organism. In some embodiments, the avian antibodies in the egg products can attach to the outer surface cell walls upon administration and prevent the organism from attaching to the cells in the mucous membranes. The microorganisms will not be able to multiply or colonize in the individual upon binding by the specific avian antibodies. The formulation can keep the microorganisms from moving down the respiratory tract and eliminate the ability to cause disease in the lower respiratory tract. By coating the antibody material on mucous membranes of the pharyngeal area, the coating can prevent the viruses or other microorganisms from infecting an individual. Delivery of the formulations described herein provide for a substantial decrease in infections in individuals and transmission of the disease caused by the viruses.

In some embodiments, the egg products are formulations that include microbial adherence inhibitors, in the form of fowl egg antibodies. In some embodiments, the egg products are whole egg material, egg yolks and/or egg whites. The egg products can include antibodies that are prepared to maintain functional antibody molecules. The microbial adherence inhibitors, e.g. avian antibodies, can be formulated for delivery to coat and protect the mucous membranes of the oropharyngeal and/or nasopharyngeal areas in individuals. The formulations can bind and neutralize viruses in the pharyngeal area of an individual. In some embodiments, the egg products are formulations for delivery as eye drops to the eyes. In some embodiments, the egg product can be formulated, for example, by protective coating, for binding and neutralizing disease-causing organisms in the stomach and intestines.

In one embodiment, the formulations are oral formulations such as a tablet, a throat lozenge, a mint, an oral spray, a mouthwash, chewing gum and the like. The oral formulations can coat and protect the oropharyngeal mucous membranes and prevent entry of the virus into the individual to reduce the risk of infection.

In one embodiment, the formulations are powdered formulations of avian egg antibodies for nasal administration by inhalation through the nasal passages to coat and protect the nasopharyngeal mucous membranes and prevent the risk of infection.

In one embodiment, the egg products can be administered to be functional in the gastrointestinal (GI) tract. The egg products may be effective for binding and neutralizing the targeted microbial organism in the GI tract, e.g. in the stomach and intestinal areas of the GI tract.

In one embodiment, the egg product may be formulated as a spray that can include the avian antibodies. The egg product may be sprayed onto the inner surface and/or the outer surface of a facemask. Coating the inner surface and/or the outer surface of a facemask can bind and/or neutralize the microbial organism emitted by an infected individual. In one embodiment, the virus expelled by an infected facemask wearer may be neutralized prior to release into the environment. In one embodiment, any droplets or aerosol particles present in the environment may be neutralize on the exterior of mask to reduce the risk of the facemask wearer from being infected by the virus particles in the air.

The present description can also include methods of preventing or reducing the infection of an individual by one or more viruses. The present description can also include methods of reducing transmission of viral disease by decreasing infectious viral particles, e.g. hot viral particles, expelled by an infected individual. The methods can include administering formulations of egg products to coat and protect the pharyngeal mucous membranes in order to neutralize the one or more viruses that cause respiratory disease.

The formulations can be directed to substantially prevent the attachment or adherence of disease-causing organisms, e.g. viruses, by inhibiting the ability of the organism to adhere to the mucous membranes of individuals and cause disease. The formulations can reduce disease transmission by preventing entry of viruses into an uninfected individual. The formulations can also reduce disease transmission by reducing infectious particles that are expelled from an infected individual. The present description includes formulations of avian antibodies that are delivered easily and economically to individuals susceptible to viral infections and to individuals harboring viral infections.

Viruses have evolved a number of different types of molecules, referred to herein as viral fusion molecules, on their surfaces which can very tightly stick to one or more types of specific molecules that are part of the host's cell surfaces. The avian antibodies described herein can be of extraordinary high specific activity which can very tightly bind to, coat, cover and obliterate these viral fusion molecules which attach themselves to their host cell's attachment sites with a lock and key type of fit to very unique chemical structures. The avian IgY immunoglobulins in the yolk tightly bind to, coat, cover and obliterate viral fusion molecules that attach themselves to their hosts.

The egg products described herein can be used as a mucosal protectant of the pharyngeal area against one or more viruses in individuals. In some embodiments, the individuals may be susceptible to the infectious disease due to potential exposure to an infected individual(s) in a workplace, home, school, or other public or private settings. In some embodiments, the individual may be immunosuppressed and is more susceptible to viral infections when exposed to an infected individual(s). In some embodiments, the individual may use the egg products for prophylactic purposes due to prolonged exposure to individuals with an unknown infection status. In some embodiments, the individual may be an infected individual and use of the egg product can reduce the number of infectious particles that are expelled by the infected individual into the environment. In some embodiments, the individual may be an infected individual and consumption of the egg products can reduce the symptoms of the infectious disease.

The avian antibodies in the egg products can be specific for the target immunogens. The target immunogens can be viral antigens from one or more target viruses that can be injected into the hens to generate the avian antibodies. The viral antigens can be virus particles, live or inactivated, viral proteins, viral macromolecules, viral fragments, and/or any other material derived from viruses. In one embodiment, virus particles can be used as immunogens to inoculate hens in order to generate the desired avian antibodies for formulation. In one embodiment, the immunogen can include all or part of a protein that can be present on the surface of the viral particles. In one embodiment, the immunogen can be all or part of the molecule that can bind and/or attach to the cellular attachment site in the individual's cells. In one embodiment, the immunogen can be the viral fusion molecule, e.g. the virus molecule that attaches to an individual's target cell.

In some embodiments, the viral antigens inoculated into the chickens are isolated and/or derived from viral particles. In some embodiments, the viral antigens used as immunogens are recombinant molecules. In some embodiments, the viral antigens, e.g. polypeptides, are expressed in recombinant expression systems. The viral antigens may be isolated from the recombinant expression systems and used as immunogens for inoculating the chickens.

In some embodiments, the viral fusion molecule can include all or part of a polypeptide fragment that is expressed on the surface of the viral particles. In one embodiment, the viral fusion molecule can be the Spike Glycoprotein S protein in SARS-CoV-2. In some embodiments, the viral antigens can include antigens from SARS-CoV-2 such as s2 spike protein, s1 spike protein, spike rbd protein, nucleocapsid, envelope and/or combinations thereof. In some embodiments, the antigens from SARS-CoV-2 can include nucleocapsid protein (GTX135357-pro), Spike RBD protein, His tag (active) (GTX01546-pro), Envelope protein, His and Avi tag (GTX01547-pro). These antigens can be purchased from, for example, GeneTex, Irvine, CA. Other membrane proteins that are immunogenic and can inhibit the binding of a viral particle to the target cells in an individual may also be used and all are within the scope of this description. In one embodiment, the viral fusion protein can also include other molecules such as sugars, lipids and the like.

Methods of preparing disease antigens, inoculating hens and generating avian antibodies specific for a disease-causing organisms are described in U.S. Pat. Nos. 9,849,175 and 10,407,494, both to Mittenesss and both incorporated herein by reference.

In some embodiments, groups of young hen chickens can be obtained such as Rhode Island Reds, White Leghorns, sex-linked hybrid crosses or other breeds suited to large egg size, high volume egg production and ease of handling which are about to reach laying age, about 16-19 weeks for chickens, on a schedule predetermined by the amount and timing of final product desired resulting in a steady continuous production stream. After a suitable period of isolation and acclimatization of about two to four weeks, each group can enter into an inoculation program using preparations of specific antigens (immunogens) such as viruses, to which an antibody is desired. In some embodiments, the cultures of microorganisms may be obtained from commercial sources such as the American Type Culture Collection (ATCC) or from wild type isolates. In some embodiments, the microorganisms may be cultured from infected individuals. The cultures may be used to isolate antigens.

The viral antigens can be prepared as immunogens and the hens may be injected intramuscularly, but preferably injected subcutaneously. In approximately four to five weeks, the average egg collected will contain copious amounts of the desired specific antibody in a readily usable and stable form. The chickens may be re-inoculated with the targeted immunogen throughout the egg laying period to maintain the high antibody level.

Batches of eggs from predetermined groups of chickens can be cracked, the contents can be separated from the shells and mixed and preferably pasteurized to eliminate potential pathogenic microorganism. Standard test procedures are used, such as ELISA, agglutination, or the like are used to the monitor the antibody activity. The typical batch is then blended with batches from groups of chickens at other average production levels resulting in abundant standardized active ingredients.

In embodiments for egg products specific to one target virus, the formulations can be derived from egg material wherein the hens are inoculated with viral antigens from the one target virus. In some embodiments, all of the hens may be inoculated with the same antigen from one virus. In some embodiments, the hens may be inoculated with different antigens from the same target virus and the egg material from all the hens may be combined.

In embodiments for egg products specific for more than one virus, the egg products can be derived from egg materials from a variety of hens that have been inoculated with viral antigens from different target viruses. The egg material specific for the different target viruses may be combined to form a formulation that is specific some or all of the target viruses.

In some embodiments, egg products with the avian antibodies that cross-react with the target virus may be used. In such embodiments, the avian antibodies may be specific for a first non-target virus and these egg products may be used against a desired target virus.

In some embodiments, the egg product can be whole eggs that include the avian antibody material. Whole egg material may be consumed by the individual wherein the avian antibodies in the egg yolk still include functional antibodies. In some embodiments, egg yolks may be consumed by the individual wherein the avian antibodies in the egg yolk still include functional antibodies. In some embodiments, the whole egg material and/or egg yolk are consumed raw by the individual. In some embodiments, the whole egg material is partially cooked, e.g. the egg white is cooked but the egg yolk is runny and/or uncooked. In some embodiments, the whole egg is cooked “over-easy” with a runny egg yolk. In some embodiments, the whole egg is poached but with a runny egg yolk.

In some embodiments, the egg material that includes the avian antibodies may be used without further purifications in the formulated egg products. In some embodiments, the avian egg material may be partially purified such as, for example, by water extractions. In some embodiments, the egg material may be substantially or fully purified to obtain the avian antibodies that bind the target disease-causing organism. The antibodies may also be purified, dried and lyophilized for storage for later use.

Dependent on the needs and specifications of the formulator and the final customer, the final formulations may include excipients or some type of innocuous additive. In some embodiments, the formulations can include excipients. A variety of excipients is known in the field and can be included in the formulations and all are within the scope of this description. The excipients can vary depending on the nature of the formulation. Excipients can include, for example, anti-adherents, binders, coatings, colors, disintegrants, flavors, glidants, lubricants, preservatives, sorbents, sweeteners, vehicles and the like. Binders can include, for example, saccharides, e.g. sucrose, lactose, starches, celluloses, xylitol, sorbitol, mannitols, gelatin, polyvinylpyrrolidone (PVP) polyethylene glycol (PEG) and the like. In some embodiments, dry binders such as cellulose, methylcellulose, PVP, PEG and the like may be added to a powder blend that includes avian antibody powder either after a wet granulation step or as part of a direct powder compression. The amounts of excipients in formulations can vary and can be dependent on the desired formulation and/or dosage.

The egg products with the avian antibodies described herein can specifically bind and inhibit a variety of viruses that can cause the infectious disease. The egg products can include avian antibodies against DNA viruses and/or RNA viruses. In some embodiments, the viruses can be, for example, Influenza viruses, Coronavirus, Henipavirus, Ebola virus, Hantaan virus, Lassa fever virus, Marburg virus, Crimean-Congo haemorrhagic fever virus, Monkeypox virus, Rift Valley Fever virus, South American haemorrhagic fever viruses, Central European tick-borne encephalitis virus, Far Eastern tick-borne encephalitis virus, Japanese encephalitis virus, Russian spring and summer encephalitis virus, Kyasanur forest disease virus, Omsk hemorrhagic fever virus and West Nile virus. In some embodiments, the formulations with the avian antibodies can be directed against viruses that include RNA viruses of the order Mononegavirales that contain single-stranded genomes that are negative sense. These viruses can include, for example, Orthomyxoviridae (which contains the influenza viruses) and Paramyxoviridae (which contains the parainfluenza viruses (PIVs), human respiratory syncytial virus (RSV), and human metapneumovirus (hMPV). The viruses inhibited by the formulations can also include, for example, Picornaviridae, rhinoviruses, enteroviruses (such as coxsackieviruses and numbered enteroviruses). In some embodiments, DNA viruses such as Adenoviridae, Parvoviridae and the like can also be inhibited.

In some embodiments, the viruses inhibited by the egg products can include, for example, viruses from the family Coronaviridae, an enveloped, positive-sense single-stranded RNA (ssRNA). These viruses can include, for example, human coronavirus (HCoV) 229E, HCoV OC43, the severe acute respiratory syndrome-associated CoV (SARS-CoV), Middle East respiratory syndrome-related coronavirus (MERS-CoV), HCoV, NL63, HCoV HKU1 and the like. In one embodiment, virus detections system in facemasks can detect SARS-CoV-1 and/or SARS-CoV-2.

In some embodiments, the avian antibodies may be directed towards antigens of influenza viruses and other microorganisms that may cause respiratory diseases in individuals. The viruses can be, for example, Orthomyxoviridae, specifically influenza, H₁N₁, H₅N₁, H₃N₂, or combinations thereof or other types of Hemagglutinin (H) and neuraminidase (N) combinations that are typically identified by an H number and an N number and their mutated strains; the Herpesviridae, specifically, Infectious Bovine Rhinotracheitis, 1 and 5; the Paramyxoviridae, specifically BRSV and PI₃; the Arteriviridae, specifically, porcine respiratory and reproductive syndrome virus (PRRSv) and the Adenoviridae, specifically Bovine adenovirus 1, 3, 5, 6, 7.

In some embodiment, the egg products may include avian antibodies that bind and neutralizes other disease-causing organisms in addition to the target viruses. The susceptibility for a secondary infection by a second disease-causing organism may be increased in individuals that are fighting a primary infection by the target virus or viruses. In some embodiments, bacteria, viruses, fungi, protozoa and the like can cause a secondary infection.

In some embodiments, the avian antibodies in the egg products may be directed against disease-causing organisms that cause secondary infections. The disease-causing organisms causing secondary infections can include other viruses as described above, and bacteria, protozoa, and the like. The disease-causing organisms that can cause secondary infections can include, for example, Gram positive cocci, Gram positive rods, Gram negative cocci, Gram negative rods and the like. Gram positive cocci can include, for example, Streptococcus pneumoniae, Streptococcus pyogenes, Streptococcus agalactiae, Staphylococcus aureus, Peptostreptococcus anaerobe. Gram positive rods can include, for example, Bacillus anthracis, Nocardia, Actinomyces anaerobe. Gram negative cocci can include, for example, Neisseria meningitidis, Moraxella catarrhalis. Gram negative rods can include, for example, Klebsiella pneumoniae, Escherichia coli, Pseudomonas aeruginosa, Acinetobacter, Burkholderia pseudomallei, Burkholderia mallei, Yersinia pestis, Francisella tularensis, Hemophilus influenzae, Bordetella pertussis, Bacteroides melaninogenicus anaerobe, Fusobacterium anaerobe, Porphyromonas anaerobe, Prevotella anaerobe, Proteus, Serratia. The disease-causing organisms can also include, for example, Mycobacterium tuberculosis, other Mycobacterium, Legionella pneumophila, Mycoplasma pneumoniae, Chlamydia trachomatis, Chlamydia psittaci, Chlamydia pneumonia, TWAR agent, Coxiella burnetii.

In one embodiment, an individual susceptible to infection by SARS-CoV-2 may also be susceptible to infections by bacteria such as Streptococcus pneumoniae, Haemophilus species, Staphylococcus aureus and Mycobacterium tuberculosis. Thus, an avian antibody formulation may include avian antibodies against SARS-CoV-2, Streptococcus pneumoniae, Haemophilus species, Staphylococcus aureus and Mycobacterium tuberculosis. Secondary infections by other potential disease-causing organisms may be alleviated by inclusion of avian antibodies against disease-causing organisms that an individual may be susceptible to when infected with the target virus or viruses.

In some embodiments, the avian antibody material can be dried into a powder. In some embodiments, the antibody material can be spray-dried powder. In some embodiments, the antibody material may include carrier materials. The antibody material may be dried prior to or after the addition of carrier materials.

Spray drying of antibody material can maintain the function of the antibodies and the ability of the antibodies to coat and protect the mucosal membranes from binding and attachment of the viral particles. Spray drying methods are known in the art and a variety of spray drying techniques may be used and are within the scope of this description. Spray drying can include atomizing of a liquid from a nozzle into a high temperature environment. The high temperature can remove the liquid in the sample. In some embodiments, the outlet air temperature for spray drying the egg material can be at a temperature below about 180° F. In one embodiment, the outlet air temperature in the spray dryer can be between about 150° F. and about 175° F. The outlet air temperature may be below about 175° F., or about 165° F.

In some embodiments, the dried egg material with the avian antibodies can include powder particles. The size of the powder particles of avian antibodies can vary and all are within the scope of this description. In some embodiments, the powder particles can include particles between about 5 microns and about 400 microns. Particles outside of this range are also within the scope of this description.

In some embodiments, the powder with the avian antibodies may be formulated into an oral formulation. In some embodiments, oral formulations can include a mint, a tablet, a throat lozenge, a gargle, a mouth rinse and the like. In some embodiments, impregnated strips with the avian antibody material can be designed to dissolve in the mouth to release antibodies and can be used to coat the upper airways with the formulation. In some embodiments, the powder with the avian antibodies may be added to a liquid such as water, milk, orange juice and the like and can be consumed by the individual by drinking.

In one embodiment, the powder with the avian antibodies can be formulated into a tablet. Formulation of powders into tablets, mints or throat lozenges are known in the art and can include the use of carrier materials and/or excipients as disclosed above. Avian antibody powders and excipients, if used, can be combined and pressed or formed into tablets. The tablets may be coated or uncoated. The tablets can be formulated, for example, as a mint to be placed in the mouth and to dissolve slowly in the mouth to coat the oropharyngeal area of an individual to act as a mucosal protectant. The tablets may also be formulated to be chewable. Chewable tablets can also release the avian antibody in the oral cavity and coat the oropharyngeal area of an individual to act as a mucosal protectant. The tablets may not be formulated for swallowing in order to enter the digestive tract. In one embodiment, tablet formulation can include a wet granulation step. In one embodiment, tablet formulation can include direct powder compression. In some embodiments, the avian antibody powder can be formulated to form a throat lozenge and may include other ingredients to lubricate and soothe irritated tissues in the mouth. In some embodiments, the egg products may be a capsule of tablet that has a protective coat surrounding the antibody material in order to be functional in the digestive tract, stomach, intestines and the like.

In some embodiments, the present description can include a nasal formulation of powder particles of the avian antibody materials to coat the nasopharyngeal area of an individual as a mucosal protectant. Nasal formulations can include a nasal powder, a nasal spray and the like. In some embodiment, the nasal formulation may be powder particles that may be administered by inhalation into the nasal passages to protect the mucous membranes of the nasopharyngeal area in an individual. In one embodiment, a straw may be used to inhale a powdered nasal formulation. The powdered particle formulation may be inhaled into both of the nasal passages of the individual, separately or simultaneously. The powder particles may include excipients. The excipients may be added prior to forming, or added as a powder, after the formation of avian antibody powder material. In some embodiments, the nasal formulation may be a nasal spray that is administered by, for example, squirting an amount of nasal spray into each of the nostrils.

In some embodiments, the formulations described herein formulations against SARS-CoV-2 to reduce and/or prevent Covid-19 in individuals. In some embodiments, avian antibodies can be generated in hens against viral antigens from SARS-CoV-2 as described above. In one embodiment, the avian antibodies generated against the SARS-CoV-2 antigens can inhibit the binding and attachment of SARS-CoV-2 to cellular attachment site, e.g. ACE2 receptor, in the individual. In some embodiments, tablets, throat lozenges, nasal inhalants and the like are used to coat the pharyngeal areas in an individual to act as a mucosal protectant against infection from SARS-CoV-2.

In one embodiment, the present description can include a kit for nasal administration. The kit can include powdered egg material containing avian antibodies in a bag such as a plastic bag. The kit may also include a scoop or other similar device to scoop a desired dose from the bag when needed by the individual administering the dose. The kit may also include a straw for inhalation of the egg powder. When desired, the appropriate dose of the egg powder is removed from the bag and placed on a hard surface. The straw can be used to inhale the egg powder into the nasopharyngeal area to act as a mucosal protectant. The egg powder can be stable without refrigeration. The egg powder can be stable in ambient temperatures even in tropical climates. The kit described herein can be advantageous as a low-cost protection against respiratory disease throughout the world.

The present description can include methods of inhibiting the ability of viruses to adhere to the mucous membranes in the pharyngeal area of humans to prevent respiratory infections and disease. The method can include administering a nasal and/or an oral formulation to coat the nasopharyngeal area and/or the oropharyngeal area in an individual. The administration may be self-administration and/or administration by others. In some embodiments, administration can include placing the oral formulations in the mouth of an individual and allowing the avian antibody material to disperse in the mouth and coat the oropharyngeal area to act as a mucosal protectant. In some embodiments, administration can include administration of nasal formulations into each of the nostrils to coat the nasopharyngeal area to act as a mucosal protectant. In some embodiments, the method can be used to inhibit SARS-CoV-2 that causes the respiratory disease Covid-19.

In one embodiment, the method may include administering avian antibody material against one virus. In one embodiment, the method may include administering avian antibody material against one or more strains of the virus. In one embodiment, the method can include administering avian antibody material against more than one virus. In one embodiment, the method can include administering avian antibody material against more than one strain of more than one virus.

The present description can include methods of reducing disease transmission within a population of individuals. The method can bind and neutralize the viruses in the oral and nasal passages in an individual. The method can include administering a nasal and/or an oral formulation to coat the nasopharyngeal area and/or the oropharyngeal area in an individual. The administration may be by self-administration and/or administration by others. In some embodiments, administration can include placing the oral formulations in the mouth of an individual and allowing the avian antibody material to disperse in the mouth and coat the oropharyngeal area to act as a mucosal protectant. In some embodiments, administration can include administration of nasal formulations into each of the nostrils to coat the nasopharyngeal area to act as a mucosal protectant. In some embodiments, the method can be used to inhibit SARS-CoV-2 that causes the respiratory disease Covid-19.

The present description can include methods of reducing disease transmission by infected symptomatic or asymptomatic individuals. The method can bind and neutralize the viruses in the oral and nasal passages in an infected individual. This can result in reducing the number of infectious viral particles expelled by the infected individual into the environment in respiratory droplets, air particles and the like. The expelled particles may have reduced number of infectious viral particles since the viral particles may be bound and neutralized by the avian antibody material. The method can include administering a nasal and/or an oral formulation to coat the nasopharyngeal area and/or the oropharyngeal area in an infected individual.

In one embodiment, the method may include administering avian antibody material against one virus. In one embodiment, the method may include administering avian antibody material against one or more strains of the virus. In one embodiment, the method can include administering avian antibody material against more than one virus. In one embodiment, the method can include administering avian antibody material against more than one strain of more than one virus, e.g. multiple strains of multiple viruses.

The avian antibody material can be delivered several times a day, e.g. as a nasal formulation and/or an oral formulation, as described herein, depending on expected exposure to provide longer term or more intense protection. In order to protect an individual during viral outbreaks, the individual susceptible to virus exposure can carry formulations containing avian antibody material. Individuals may carry the formulations and self-administer the formulations multiple times in a day to decrease the chance of infection from a virus or viruses. Individuals could easily apply the formulations to their pharyngeal areas prior to expected exposure by the method of administering the formulations. These avian antibodies would serve as a mucosal protectant to prevent the adherence and replication of the targeted virus or other microorganism in the individual.

The avian antibodies against a virus may be prepared on a yearly or other periodic time frame as new strains of the virus emerge. The avian antibodies administered may be based on the circulating strains of the viruses. As circulating strains change or the threat of a new, pandemic virus appears; simply immunizing new hens with the newest strain or strains of interest may update the product. The birds may begin to deposit antibodies into their eggs specific to the newest strain or strains in as little as two weeks after immunization.

### EXAMPLE

In-Vitro Studies of Chicken-Derived IgY Against SARS-CoV-2

Recombinant spike protein from SARS-CoV-2 was expressed and purified. Chickens were inoculated with the spike protein.

The chicken-derived IgY samples labeled 052120, 060820, 061520 and 062220 were assessed for their ability to neutralize SARS-CoV-2 virus using a PRNA assay. Briefly, the antibodies were diluted 1:10 in culture medium and incubated with 200 plaque forming units (PFUs) of the Washington Strain of SARS-CoV-2 (USA-WA1/2020). SARS-CoV-2 was diluted in supplemented DMEM to appropriate concentration. Virus was then added to antibody samples and allowed to incubate for 1 hour at 37° C. and 5% CO2. After incubation, viral plaque assay was conducted to quantify viral titers. 12-well plates were previously seeded with Vero cells (ATCC CCL-81) at a density of 2E5 cells per well. Media was aspirated from plates and virus-antibody samples were transferred to wells, one sample per well. Plates were inoculated for 1 hour at 37° C. and 5% CO2. After infection, a 1:1 overlay consisting of 0.6% agarose and 2× Eagle's Minimum Essential Medium without phenol red (Quality Biological, 115-073-101), supplemented with 10% fetal bovine serum (FBS) (Gibco, 10,437,028), non-essential amino acids (Gibco, 11140-050), 1 mM sodium pyruvate (Corning, 25-000-Cl), 2 mM L-glutamine, 1% P/S was added to each well. Plates were incubated at 37° C. for 48 hours. Cells were fixed with 10% formaldehyde for 1 hour at room temperature. Formaldehyde was aspirated and the agarose overlay was removed. Cells were stained with crystal violet (1% CV w/v in a 20% ethanol solution). Viral titer of SARS-CoV-2 was determined by counting the number of plaques.

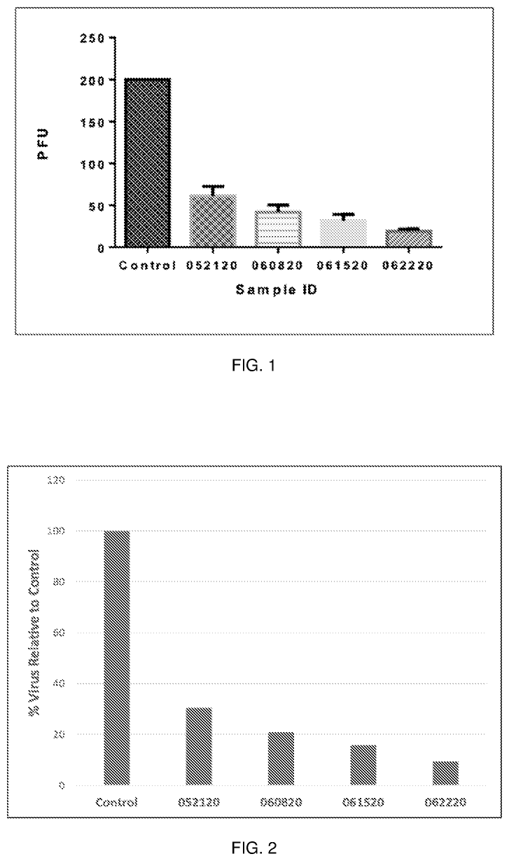

FIG. 1 depicts the absolute neutralization of virus observed in this assay at 1:10 dilution of the antibodies. Control refers to virus count in the absence of the antibody. Sample 052120 is chicken IgY antibody from chickens that have not been inoculated with the spike protein. Samples 060820, 061520 and 062220 are chicken IgY antibodies wherein the chickens have been inoculated with spike protein. FIG. 2 depicts the same data as relative percentage of neutralization observed in the context of the four antibodies as compared to the virus control.

Although the present invention has been described with reference to preferred embodiments, workers skilled in the art will recognize that changes may be made in form and detail without departing from the spirit and scope of the invention.

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. A method of decreasing the transmission rate of an infectious disease within a population of individuals comprising:
providing egg products comprising egg material from eggs to individuals in a population susceptible to or having the infectious disease, wherein the eggs are from female birds inoculated with one or more disease antigens from microbial organisms causing the infectious disease, wherein the egg material comprises one or more avian antibodies that specifically bind and neutralize the microbial organisms in the individuals when consumed by the individuals.

24. The method of claim 23, wherein the female birds are egg laying birds on egg farms.

25. The method of claim 23, wherein the egg material is formulated into a formulation comprising egg powder.

26. The method of claim 23, wherein the egg product is a nasal inhalant, a nasal spray, mouth wash, oral spray, oral tablet, a mint, chewing gum, a throat lozenge and combinations thereof.

27. The method of claim 23, wherein the microbial organisms are bacteria, viruses, fungi, protozoa, worms or combinations thereof.

28. The method of claim 23, wherein the microbial organism is a virus and wherein the one or more avian antibodies prevent a viral fusion molecule in the virus from attaching to the cellular attachment site of cells in the individual.

29. The method of claim 28, wherein the cellular attachment site in the cells comprises the ACE2 receptor in the individual.

30. The method of claim 23, wherein the avian antibodies are directed against an antigen from SARS-CoV-2, wherein the antigen comprises s2 spike protein, s1 spike protein, spike rbd protein, nucleocapsid, envelope or combinations thereof.

31. The method of claim 23, wherein the avian antibodies reduce the susceptibility or symptoms to the disease upon consumption of the egg product.

32. (canceled)

33. The method of claim 23, wherein the egg product is consumed by the individuals at least once a day.

34. (canceled)

35. (canceled)

36. The method of claim 23, wherein the egg product comprises egg material separated from the egg shells and at least part or all of the egg yolk in the egg material comprises non-denatured antibody material when consumed by the individual.

37. The method of claim 23, wherein the egg material is formulated as an egg product for consumption by the individuals and wherein the formulation comprises the one or more antibodies that specifically bind and neutralize the microbial organisms when consumed by the individuals.

38. The method of claim 23, wherein the egg product is formulated for application to the pharyngeal area, or the nasopharyngeal area, or the oropharyngeal area of the individuals.

39. (canceled)

40. (canceled)

41. (canceled)

42. The method of claim 27, wherein the viruses comprises Influenza viruses, Coronaviruses, Henipavirus, Ebola virus, Hantaan virus, Lassa fever virus, Marburg virus, Crimean-Congo haemorrhagic fever virus, Monkeypox virus, Rift Valley Fever virus, South American haemorrhagic fever viruses, Central European tick-borne encephalitis virus, Far Eastern tick-borne encephalitis virus, Japanese encephalitis virus, Russian spring and summer encephalitis virus, Kyasanur forest disease virus, Omsk hemorrhagic fever virus, West Nile virus, human respiratory syncytial virus (RSV), human metapneumovirus (hMPV), Picornaviridae, rhinoviruses, enteroviruses, viruses from the family Coronaviridae, or combinations thereof.

43. (canceled)

44. The method of claim 27, wherein the virus comprises SARS-CoV-2.

45. The method of claim 23, wherein the consumption of the egg product reduces susceptibility to Covid-19 or prevents Covid-19.

46. The method of claim 23, wherein the egg products are provided to all of the individuals in a population residing and/or working in a region with an outbreak of the infectious disease.

47. The method of claim 23, wherein the providing the egg products disrupts and/or decreases the transmission chain of the infectious disease from infected individuals in a population to uninfected individuals in a population.

48. The method of claim 23, wherein the individuals consuming the egg products shed neutralized microbial organisms.

49. (canceled)

50. (canceled)

51. (canceled)
